(19) United States
(12) Patent Application Publication
(10) Pub. No.: US 2022/0061707 A1
(43) Pub. Date: Mar. 3, 2022

(54) GLUCOSE EXPOSURE SYSTEMS AND PROCESSES

(71) Applicant: TT1 Products, Inc., Atlanta, GA (US)

(72) Inventors: Harold Philpott Southerland, III, Atlanta, GA (US); Todd Furneaux, Atlanta, GA (US); Juan Pablo Frias, Atlanta, GA (US); Roger Steven Mazze, Excelsior, MN (US)

(21) Appl. No.: 17/008,214

(22) Filed: Aug. 31, 2020

(51) Int. Cl. A61B 5/145; A61B 5/00; G16H 40/67

(52) U.S. Cl. CPC A61B 5/14532; A61B 5/0004; A61B 5/0022; A61B 5/7203; A61B 5/002; A61B 5/742; G16H 40/67; A61B 5/1451; A61B 5/7282

(57) ABSTRACT

The present systems and processes generally relate to measuring glucose exposure, determined, for example, on an hourly basis. This glucose exposure, in at least one embodiment, represents a current glucose level over the span of given period (e.g., a portion of 24 hours). In at least one embodiment, the present systems and processes display this glucose exposure in relation to a glucose target or limit.

### BACKGROUND

Average glucose monitoring is typically measured on a 24 hour basis and over time (e.g., what is a person's average 24 hour glucose, averaged over weeks or months). This average glucose data may be used to determine a number of factors. Such factors include, but are not limited to, diabetes, pre-diabetes, insulin resistance, etc. While controlling/understanding average 24 hour glucose (averaged over time), more granular glucose monitoring may help control glucose throughout a day and lead to additional physiological advantages and/or assist with weight or training targets.

Thus, there is a long-felt but unresolved need for a system or process that continuously monitors and reports glucose levels in the context of overall glucose targets or limits.

### BRIEF SUMMARY OF THE DISCLOSURE

Briefly described, and according to one embodiment, aspects of the present disclosure generally relate to systems and processes for measuring glucose exposure.

The present disclosure generally relates to systems and processes for measuring glucose exposure, determined, for example, on an hourly basis. This glucose exposure, in at least one embodiment, represents a current glucose level of a user over the span of given period (e.g., a portion of 24 hours). In at least one embodiment, the present systems and processes display this glucose exposure in relation to a glucose target or limit.

For example, the present systems and processes may determine (or receive) a 24 hour glucose target or limit and determine a glucose limit or target for a particular hour (e.g., at 10:00 AM, a user should have a glucose exposure of 1000 mg/dl, where 1000 mg/dl is a summation of glucose from previous hours in the day). Continuing with this example, the present systems and methods may determine a glucose exposure for the user of 1010 mg/dl by summing the glucose exposure of the user for each hour until 10:00 AM. The present systems and methods, in this example, may display the current glucose exposure (e.g., 1010 mg/dl) as a percentage or comparison of the glucose target or limit of 1000 mg/dl, potentially indicating that the user to trending towards a glucose level that is over the glucose target or limit for the 24 hour period.

According to a first aspect, the glucose exposure process may include receiving a glucose exposure limit per hour, determining a target glucose for a particular hour by multiplying the glucose exposure limit per hour by a numerical representation of the particular hour, determining a glucose exposure for the particular hour by: determining whether a receiver is connected to a sensor; if the receiver is connected to the sensor: A) receiving, via the receiver and from the sensor, obfuscated glucose information derived from a filament interacting with tissue fluid at a fixed interval; B) deobfuscating the glucose data; and C) determining the glucose exposure for the particular hour based on the deobfuscated glucose information; if the receiver is not connected to the sensor: A) determining an average glucose exposure for the particular hour based on stored information; and B) determining the glucose exposure for the particular hour based on the average glucose exposure for the particular hour; determining a total of the glucose exposure by adding the glucose exposure for the particular hour to a summation of glucose exposure for time intervals preceding the particular hour, and displaying the running total of the glucose exposure as a proportion of the target exposure for the particular hour.

According to a second aspect, the glucose exposure process of the first aspect or any other aspect, wherein determining the average glucose exposure for the particular hour based on stored information includes averaging a glucose exposure for an hour immediately preceding the particular hour and an hour immediately following the particular hour.

According to a third aspect, the glucose exposure process of the first aspect or any other aspect, wherein determining the average glucose exposure for the particular hour based on stored information includes: A) retrieving stored glucose exposure information for the particular hour; and B) averaging the stored glucose exposure information for the particular hour.

According to a fourth aspect, the glucose exposure process of the third aspect or any other aspect, wherein the stored glucose exposure information is stored in memory at a cloud-based device.

According to a fifth aspect, the glucose exposure process of the first aspect or any other aspect, wherein the fixed interval is about 1 minute.

According to a sixth aspect, the glucose exposure process of the first aspect or any other aspect, wherein the fixed interval is about 15 minutes.

According to a seventh aspect, the glucose exposure process of the first aspect or any other aspect, wherein the fixed interval is about 60 minutes. According to an eighth aspect, the glucose exposure process includes receiving, at a receiver and from a glucose sensor, obfuscated glucose information derived from a filament interacting with tissue fluid on a particular interval, deobfuscating the glucose information, determining a glucose exposure for a particular hour based on the glucose information at the particular interval, determining a target glucose exposure for the particular hour by multiplying a glucose exposure limit per hour by a numerical representation of the particular hour, and displaying the glucose exposure for the particular hour as a proportion of the target glucose exposure for the particular hour.

According to a ninth aspect, the glucose exposure process of the eighth aspect or any other aspect, the process further including receiving one or more additional obfuscated glucose information derived from the filament interacting with interstitial fluid on the particular interval during the particular hour.

According to a tenth aspect, the glucose exposure process of the ninth aspect or any other aspect, the process further including deobfuscating the additional obfuscated glucose information.

According to an eleventh aspect, the glucose exposure process of the tenth aspect or any other aspect, wherein the deobfuscated glucose information and the deobfuscated additional glucose information includes one or more glucose measurements.

According to a twelfth aspect, the glucose exposure process of the eleventh aspect or any other aspect, wherein determining the glucose exposure for the particular hour includes averaging the one or more glucose measurements.

According to a thirteenth aspect, the glucose exposure process of the twelfth aspect, or any other aspect, wherein determining the glucose exposure for the particular hour includes adding the average of the one or more glucose measurements to a summation of averages of glucose measurements for time intervals preceding the particular hour in a 24-hour time span.

According to a fourteenth aspect, the glucose exposure process of the thirteenth aspect, or any other aspect, wherein the averages of glucose measurements for time intervals preceding the particular hour include at least one average glucose measurement based on stored information.

According to a fifteenth aspect, the glucose exposure process of the fourteenth aspect, or any other aspect, wherein the at least one average glucose measurement includes an average of average glucose measurements for an hour immediately preceding the particular hour and an hour immediately following the particular hour.

According to a sixteenth aspect, the glucose exposure process of the fifteenth aspect, or any other aspect, wherein the at least one average glucose measurement includes an average of stored glucose exposure information for the particular hour for stored 24-hour time spans.

According to a seventeenth aspect, the glucose exposure process of the sixteenth aspect, or any other aspect, wherein the fixed interval is about 1 minute.

According to an eighteenth aspect, the glucose exposure process of the sixteenth aspect, or any other aspect, wherein the fixed interval is about 15 minutes.

According to a nineteenth aspect, the glucose exposure process of the sixteenth aspect, or any other aspect, wherein the fixed interval is about 60 minutes.

These and other aspects, features, and advantages of the claimed invention(s) will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE FIGURES

The accompanying drawings illustrate one or more embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1 is a diagram of an exemplary glucose exposure system, according to one embodiment of the present disclosure;

FIG. 2A is a flow chart of an exemplary glucose exposure process, according to one embodiment of the present disclosure;

FIG. 2B is a flow chart of an exemplary target glucose exposure determination process, according to one embodiment of the present disclosure;

FIG. 3A is a flow chart of an exemplary glucose exposure process, according to one embodiment of the present disclosure;

FIG. 3B is a flow chart of an exemplary target glucose exposure calculation process, according to one embodiment of the present disclosure; and

FIG. 3C is a flow chart of an exemplary glucose exposure for a particular hour determination process, according to one embodiment of the present disclosure.

### DETAILED DESCRIPTION

For the purpose of promoting an understanding of the principles of the present disclosure, reference will now be made to the embodiments illustrated in the drawings and specific language will be used to describe the same. It will, nevertheless, be understood that no limitation of the scope of the disclosure is thereby intended; any alterations and further modifications of the described or illustrated embodiments, and any further applications of the principles of the disclosure as illustrated therein are contemplated as would normally occur to one skilled in the art to which the disclosure relates. All limitations of scope should be determined in accordance with and as expressed in the claims.

Whether a term is capitalized is not considered definitive or limiting of the meaning of a term. As used in this document, a capitalized term shall have the same meaning as an uncapitalized term, unless the context of the usage specifically indicates that a more restrictive meaning for the capitalized term is intended. However, the capitalization or lack thereof within the remainder of this document is not intended to be necessarily limiting unless the context clearly indicates that such limitation is intended.

### Overview

In various embodiments, the present systems and processes determine glucose exposure for a particular user on an hourly basis. To do so, in at least one embodiment, the systems and processes determine average glucose for the user for each hour, then sum the average glucose per hour over the number of hours currently passed in a given day to determine a glucose exposure for the current time/hour (e.g., sums the average glucose from 12:00 AM-1:00 AM, 1:00 AM-2:00 AM, 2:00 AM-3:00 AM, and 4:00 AM-5:00 AM to determine a glucose exposure at 5:00 AM).

In order to determine an average glucose for an hour, in at least one embodiment, the disclosed systems and processes: a) receive glucose data (e.g., a reading of current glucose levels of a user) from a sensor at predetermined/fixed intervals (e.g., 15 minutes); and b) averages the received glucose data over an hour (e.g., averages four glucose readings taken at 15 minute intervals over the hour).

In at least one embodiment, the present systems and processes display current glucose exposure for a particular hour as a percentage (or proportion) of a glucose target or limit for the day and/or hour (or a limit/target for a day thus far). In some embodiments, the systems and processes may create alerts based on current glucose exposure. For example, if a user's current glucose exposure is over the target or limit for the hour (e.g., thus far in the day), then the systems and processes may recommend that the user embark on glucose reducing or limiting actions (e.g., go for a walk, eat a low carb lunch, etc.). In this way, the systems and processes may enable a user to tweak habits or actions to influence glucose exposure during a day.

In at least one embodiment, the systems and processes may “backfill” glucose data for times when a sensor (e.g., that reads a user's glucose) is disconnected or glucose readings are otherwise unavailable (e.g., data could be corrupted, unavailable, or otherwise unusable) via one or more processes or mechanisms discussed herein.

For example, in one embodiment, a user (or the system) may set a target glucose limit of 1200 units of measurement (such as, e.g., milligrams per deciliter) per day. Based on the target glucose limit, the glucose exposure system may determine at a given time the target glucose limit for that given time, as well as the user's glucose exposure. Continuing with the above example, if the day begins at midnight (00:00 AM) and goes for 24 hours, then the glucose exposure limit per hour would be 50 mg/dl. If the user checks his glucose exposure at 8:00 AM, the target glucose exposure would be 400 mg/dL, and the glucose exposure system would display the user's glucose exposure (e.g., 390 mg/dl based on sensor readings, as discussed herein) compared to the target glucose for 8:00 AM, to indicate to the user if the user was below, at, or above the target glucose exposure limit for 8:00 AM.

In another example, in one embodiment, a user (or the system) may set a glucose exposure limit per hour of 90 mg/dL. Based on the glucose exposure limit per hour, the system 100 may determine at a given time the target glucose exposure for that given time, as well as the user's glucose exposure. Still continuing with the above example, if the day begins at midnight (00:00 AM), and the current time is 11:00 AM, the target glucose exposure would be 990 mg/dL. Still continuing with the above example, the system 100 would display the user's glucose exposure (e.g., 1000 mg/dL based on sensor readings, as discussed herein) compared to the target glucose exposure for 11:00 AM, to indicate to the user if the user was below, at, or above the target glucose exposure limit for 11:00 AM.

### Exemplary Embodiments

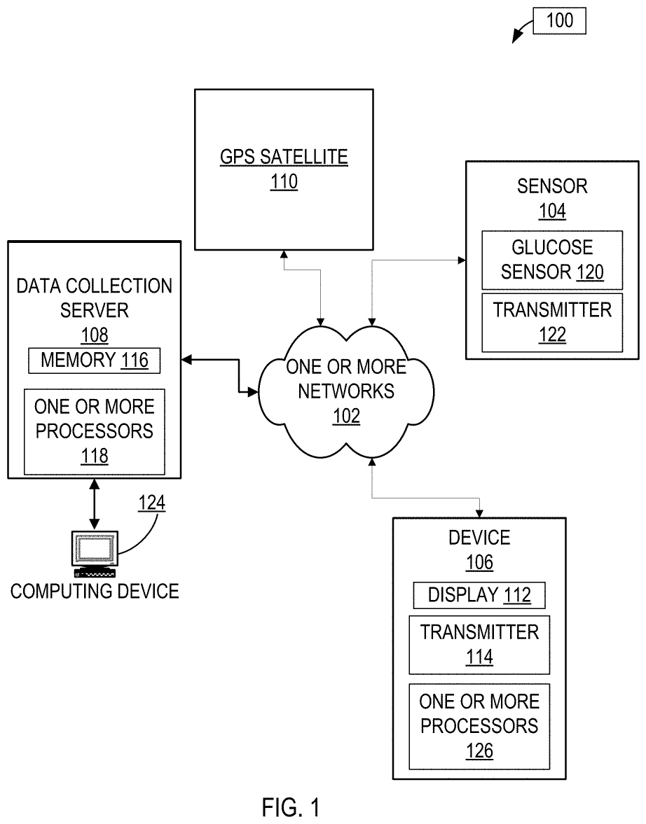

Referring now to the figures, for the purposes of example and explanation of the fundamental processes and components of the disclosed systems and methods, reference is made to FIG. 1, which illustrates an exemplary system diagram 100 of one embodiment of a glucose exposure system. As will be understood and appreciated, the exemplary diagram 100 shown in FIG. 1 represents merely one approach or embodiment of the present system, and other aspects are used according to various embodiments of the present system.

As shown in FIG. 1, according to one embodiment, the system 100 may include one or more networks 102, a sensor 104, a device 106, a data collection server 108, and a GPS satellite 110.

In various embodiments, the sensor 104 may include a glucose sensor 120 and a transmitter 122. In one or more embodiments, the glucose sensor 120 may be a filament that interacts with a user to derive glucose data. In at least one embodiment, the glucose sensor 120 may interact with interstitial fluid or other bodily fluid of the user to derive the glucose data. In several embodiments, the glucose sensor 120 may interact with the interstitial fluid or other bodily fluid of the user at a particular interval.

In multiple embodiments, the sensor 104 may also include a transmitter 122. In many embodiments, the transmitter 122 may transmit the derived glucose data from the sensor 104 to the data collection server 108 or device 106, via the one or more networks 102. In at least one embodiment, the transmitter 122 may transmit via Bluetooth radio, near-field communication (NFC), and other similar wireless communication tools.

In various embodiments, the sensor 104 receives an electronic signal from the glucose sensor 120 that indicates the amount of glucose in the interstitial fluid. In many embodiments, the sensor 104 measures the electronic signal (e.g., voltage change or the like) from the glucose sensor 120, and, from that measurement, the sensor 104 determines how much glucose is present, which the sensor 104 reads as glucose data. In one embodiment, the glucose data may include an amount of glucose in the user's interstitial fluid. In many embodiments, the amount of glucose may be measured in a unit of measurement (e.g., milligrams per deciliter). In one or more embodiments, the sensor 104 obfuscates the glucose data before transmitting the obfuscated glucose data, via the transmitter 122, to the device 106 or data communication server 108. In at least one embodiment, the sensor 104 may obfuscate the glucose data by encryption, one or more hashing algorithms, or other similar processes.

In at least one embodiment, the sensor 104 may receive an electronic signal from the glucose sensor 120 that indicates the amount of glucose in the interstitial/tissue fluid of the user. In some embodiments, the sensor 104 may measure the electronic signal, but may not translate the electronic signal into a corresponding amount of glucose present. In one or more embodiments, the sensor 104 instead may obfuscate the raw electronic signal data by encryption, one or more hashing algorithms, or other similar processes, and transmit the raw electronic signal data to the device 106 for translation into corresponding milligrams per deciliter. In many embodiments, the device 106 may receive (e.g., via a receiver or radio) and deobfuscate the electronic signal data, or transmit the obfuscated electronic signal data to the data connection server 108 to be deobfuscated. In some embodiments, once the electronic signal data is deobfuscated, the device 106 or data connection server 108 may read the electronic signal data and determine, from the electronic signal data, the amount of glucose in the user's interstitial fluid (in milligrams per deciliter).

In a further embodiment, the sensor 104 may also create a timestamp when receiving the electronic signal from the glucose sensor 120, and may associate the timestamp with the received electronic signal or measured voltage change (or other electronic data). In multiple embodiments, the timestamp data may be included in the glucose data that is obfuscated and transmitted to the device 106. In one or more embodiments, the sensor 104 may associate an identifier with the received electronic signal from the glucose sensor 120. In at least one embodiment, the sensor 104 may include the identifier in the glucose data, and obfuscate and transmit the identifier, along with the glucose data, to the device 106.

In several embodiments, the device 106 may be a mobile device, tablet, smart watch, laptop, web application, or similar devices. In one or more embodiments, the device 106 may be wearable by the user. In at least one embodiment, the device 106 may include a display 112, a transmitter 114, and one or more processors 126. In many embodiments, the display 112 may display the user's glucose exposure data, target glucose exposure data, a comparison between the user's glucose exposure data and target glucose exposure data, and/or other data related thereto.

In multiple embodiments, the device 106 may receive, via one or more radios, the glucose data, obfuscated or deobfuscated, from the sensor 104 or the data collection server 108, via the one or more networks 102. In at least one embodiment, the transmitter 114 may transmit via Bluetooth radio, NFC, and other similar wireless communication tools. In many embodiments, one or more radios on the device 106 may receive from the sensor 104 or data communication server 108 via Bluetooth radio, NFC, and other similar wireless communication tools. In various embodiments, the data collection server 108 may include memory 116 and one or more processors 118. In at least one embodiment, the memory 116 may include a storage database. In some embodiments, the memory 116 may store historical deobfuscated glucose data, along with associated time stamps, identifiers, and other associated data, for the user. In some embodiments, the data collection server 108 may retrieve the historical deobfuscated glucose data from the memory 116 for determining missing glucose exposure data.

In many embodiments, the data collection server 108 may be operatively connected to a computing device 124.

In one or more embodiments, the data collection server 108 and/or device 106 may receive the obfuscated glucose data from the sensor 104. In several embodiments, the data collection server 108 and/or device 106 may receive the obfuscated glucose data from the sensor 104 at a particular interval. In at least one embodiment, the particular interval may be a range of time from one second to one hour. For example, the data collection server 108 and/or device 106 may receive obfuscated glucose data from the sensor 104 every second, or may receive the obfuscated glucose data from the sensor once per hour, or any other interval therebetween.

In multiple embodiments, the sensor 104 may collect glucose data from the glucose sensor 120 over a predetermined interval, but, instead of transmitting each glucose data upon receiving the electronic signal from the glucose sensor 120, the sensor 104 may store the glucose data and batch the glucose data for transmitting. In many embodiments, the sensor 104 may transmit a batch of glucose data to the device 106 or data collection server 108 after a specific amount of time or after a specific amount of glucose data has been received from the glucose sensor 120. For example, in this embodiment, the glucose sensor 120 may send the sensor 104 the glucose data at a constant rate (e.g., one per second), but the sensor 104 may collect multiple glucose data from the glucose sensor 120 and only transmit the glucose data to the device 106 or data collection server 108 once the sensor 104 has received a specific amount of glucose data (e.g., every five, ten, or twenty glucose data) from the glucose sensor 120 (e.g., in a batch). In one embodiment, the predetermined interval may be the amount of time between the glucose sensor 120 sending readings to the sensor 104.

In many embodiments, the data collection server 108 and/or device 106 may store the obfuscated glucose data in the memory 116. In several embodiments, the device 106, utilizing the one or more processors 126, and/or the data collection server 108, utilizing the one or more processors 118, may deobfuscate the obfuscated glucose data. In one or more embodiments, once the obfuscated glucose data is deobfuscated, the (deobfuscated) glucose data may be stored in the memory 116 and/or in the device 106 (as will be understood, the device 106 may include local memory/storage). In at least one embodiment, the glucose data may be stored in the memory 116 and/or the device 106 for a certain amount of time, including, but not limited to, ninety days.

In various embodiments, the system 100 may also include a GPS satellite 110. In one or more embodiments, the GPS satellite 110 may be utilized to track the location of the device 106. In many embodiments, the system 100 may utilize the location of the device 106 as movement data for the user, and calculate a distance traveled by the user based on changing locations of the device 106. In at least one embodiment, the glucose disclosure system may also calculate the speed of the user by dividing the distance traveled by the user by the time it took for the user to travel the distance.

According to particular embodiments, the device 106 may include any suitable additional components, such as, but not limited to, a gyroscope, accelerometer, heart rate sensor, pulse oximeter, etc. In one embodiment, the device 106 and/or data collection service 108 may determine a step count or other suitable data for a user wearing the device 106.

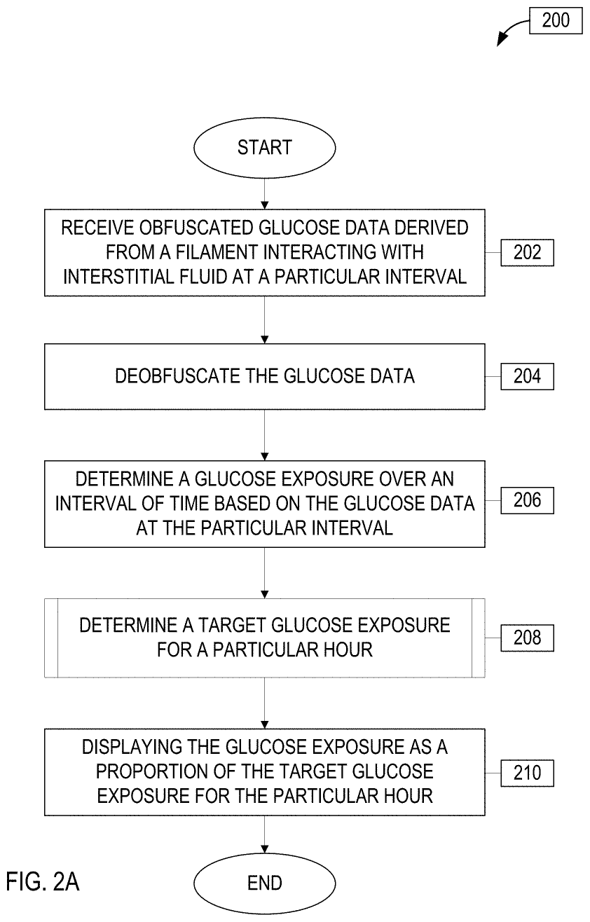

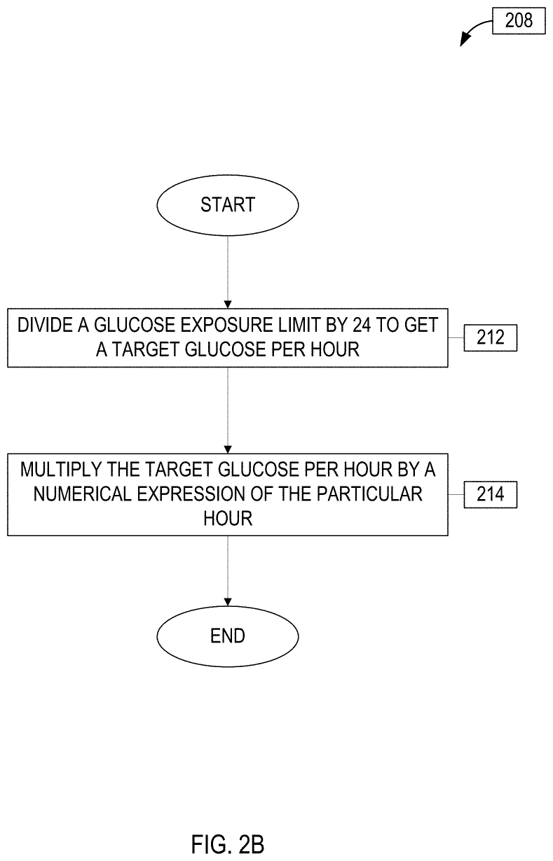

As shown in FIG. 2A, an exemplary glucose exposure process 200 is described, according to one embodiment of the present disclosure. In various embodiments, a user may first connect the sensor 104 to the user's body, such that the glucose sensor 120 is interacting with the interstitial fluid or otherwise determining a level of glucose within the patient's blood.

According to one embodiment, at step 202 of process 200, the system 100 may receive the obfuscated glucose data from the transmitter 122 of the sensor 104 at a particular interval. In at least one embodiment, the obfuscated glucose data is derived from the glucose sensor 120 interacting with the user, and specifically, with the user's interstitial fluid. In one or more embodiments, the system 100 may receive the obfuscated glucose data at the device 106 or the data collection server 108.

In multiple embodiments, the particular interval is a time interval by which the system 100 receives the obfuscated glucose data from the sensor 104. In many embodiments, the particular interval may one second, or may be one day, or any time therebetween. For example, in one embodiment, the particular interval may be 15 minutes, 30 minutes, 1 hour, 1 day, etc.

In several embodiments, the particular interval may be the time interval between the glucose sensor 120 transmitting readings to the sensor 104. In this embodiment, when the sensor 104 receives a reading of glucose data from the glucose sensor 120, the sensor 104 may also record a timestamp and associate the time stamp with the received glucose data. Continuing in this embodiment, the sensor 104 may thereafter transmit the glucose data and associated time stamp to the device 106 and/or data connection server 108.

At step 204, in various embodiments, the system 100 may deobfuscate the obfuscated glucose data received from the sensor 104. In one or more embodiments, the device 106 or the data collection server 108 may deobfuscate the obfuscated glucose data. As will be understood from discussions here, the sensor 104 may obfuscate glucose data via encryption, hashing, steganography, etc. In some embodiments, once the device 106 receives the obfuscated (or encrypted) glucose, the device 106 may deobfuscate, decrypt, or otherwise decode the glucose data. In at least one embodiment, the device 106 may transmit the obfuscated to the data collection server 108 for deobfuscation.

At step 206, in multiple embodiments, the system 100 may determine a glucose exposure over an interval of time based on the glucose data at the particular time interval. In several embodiments, the glucose exposure over the interval of time may be an average of the received glucose data at the particular interval over the course of the interval of time. In one or more embodiments, the interval of time may be the same amount of time as the particular interval, or may be a longer amount of time such that the glucose exposure may be based on more data. In at least one embodiment, the interval of time may be fifteen, thirty, or sixty minutes, a number of hours (see example below regarding a running total), or some other amount of time. For example, in one embodiment, the particular interval may be fifteen minutes, and the interval of time may be sixty minutes, such that the system 100 receives glucose data four times within the interval of time. Continuing with this example, the received glucose data over the sixty minute interval of time may be 90, 92.5, 97.5, and 100 (in units of measurement), which averages to a glucose exposure of 95 units of measurement over the sixty minute interval of time. In some embodiments, the system 100 may utilize a median determination rather than an average determination to determine the glucose exposure over an interval of time (or another other average determination herein). For example, in one embodiment, using the same numbers as the previous example, the system 100 may determine that the median glucose exposure is 95 units of measurement (average of the two middle numbers of 92.5 and 97.5) over the sixty minute interval of time.

In a further embodiment, the system 100 may calculate a running total of glucose exposure through a twenty-four hour day by adding the determined glucose exposures over the intervals of time (or a single interval of time might be the time of the running total) throughout the twenty-four hours in a day. For example, in one embodiment, if the interval of time is sixty minutes, and the twenty-four hour day begins at midnight (00:00 AM), the system 100 may add each glucose exposure over the interval of sixty minutes over the course of the twenty-four hour day, so that, at a particular hour (e.g., 9:00 AM), the system 100 may determine the total glucose exposure for the user for the day at 9:00 AM.

In an alternate embodiment, the system 100 may utilize a weighted average for determining the glucose exposure over the interval of time. In this alternate embodiment, the system 100 may give more weight to the glucose data received closer to the end of the interval of time and less weight to the glucose data received nearer to the beginning of the interval of time, so that the glucose exposure over the interval of time is closer to the current glucose exposure at the end of the interval of time. For example, in this alternate embodiment, if the system 100 received, in order, the glucose data at the particular interval of 90, 92.5, 97.5, and 100 (in units of measurement such as, e.g., in milligrams) over the interval of time, the weighted average may be greater than the actual average 95 of the glucose data.

As described in step 208, in various embodiments, the system 100 may determine a target glucose exposure for a particular hour. In one or more embodiments, and as shown in more detail in FIG. 2B, the target glucose exposure for the particular hour may be the amount of glucose exposure the user is trying to attain for the particular hour. As discussed in more detail below, in some embodiments, step 208 includes dividing a daily glucose exposure limit by 24 to get a glucose exposure limit per hour (step 212) and multiplying the glucose exposure limit per hour by a numerical expression of the particular hour (step 214). In some embodiments, the target glucose exposure for the particular hour may be a limit of glucose exposure that the user is trying not to exceed. In at least one embodiment, the particular hour may be a specific time during a twenty-four hour period. For example, in one embodiment, the particular hour may be 9:00 AM.

As described in step 210, in multiple embodiments, the system 100 may display the glucose exposure as a proportion of the target glucose exposure for the particular hour. In at least one embodiment, the system 100 may compare the running total of the glucose exposure for the user at the particular hour to the target glucose exposure for the particular hour. In an alternate embodiment, the system 100 may compare the glucose exposure over the interval of time to the glucose exposure limit per hour.

For example, in several embodiments, if the interval of time is sixty minutes, then the system 100 will determine the glucose exposure of the user every sixty minutes. Continuing with the example, in some embodiments, the running total of glucose exposure at a particular hour may be the sum of the determined glucose exposure data from the previous intervals of time for the day. Still continuing with this example, in many embodiments, if the interval of time is sixty minutes, the running total of glucose exposure at 10:00 AM may be the sum of the determined glucose exposures from 1:00 AM, 2:00 AM, 3:00 AM . . . 10:00 AM. Still continuing with this example, in one or more embodiments, if the previous determined glucose exposures for the day were 63 (00:00 AM), 60, 65, 73, 80, 84, 88, 90, 93, 95, and 97 (10:00 AM), then the running total of the glucose exposure at 10:00 AM is 888 units of measurement of glucose exposure. Continuing with this example, in one embodiment, if the target glucose exposure for 10:00 AM is 910 units of measurement of targeted glucose exposure, the system 100 may display 888 units of measurement of glucose exposure divided by 910 units of measurement of targeted glucose exposure. In a further embodiment, the system 100 may display the proportion of the glucose exposure to the target glucose exposure as a percentage.

In various embodiments, the system 100 may determine a 24-hour average glucose for the user. In many embodiments, the system may calculate the 24-hour average glucose by averaging the user's determined glucose exposure data from the previous 24-hour period. In some embodiments, the 24-hour average glucose may be a rolling average such that the 24-hour average glucose may be recalculated once an hour or once every interval of time in which the glucose exposure is determined. For example, in one embodiment, the 24-hour average glucose at 11:00 AM may be an average of the determined glucose exposure data for the previous 24 hours (e.g., from about 11:00 AM previous day to 11:00 AM current day), while the glucose exposure at the particular hour (11:00 AM) may be the sum of the glucose exposure data from midnight of the current day to 11:00 AM of the current day (eleven hours). In at least one embodiment, the system may display the 24-hour average glucose. In one or more embodiments, the system may compare the current 24-hour period to an immediately preceding 24-hour average glucose. In one embodiment, the system may display the difference between the current 24-hour average glucose to the immediately preceding 24-hour average glucose as a percentage. In some embodiments, system 100 may store the 24-hour average glucose determinations for previous days (e.g., the 24-hour average glucose determination from midnight (00:00 AM) to the next midnight (24:00) to be utilized in additional calculations.

In several embodiments, the system 100 may determine a seven-day average glucose for the user. In some embodiments, the system may calculate the seven-day average glucose by averaging the user's determined glucose exposure data from the previous seven-day period. In one or more embodiments, the system 100 may average the determined 24-hour average glucose for each of the preceding seven days to determine the seven-day average glucose. In at least one embodiment, the system 100 may display the seven-day average glucose. Similarly, in many embodiments, the system 100 may determine an average glucose for any time period (e.g., one month, one year), by averaging 24-hour average glucose determinations or seven-day average glucose determinations, or other similar glucose exposure calculations. In one embodiment, the system 100 may determine a median to calculate the 24-hour average glucose and/or seven-day average glucose.

Turning now to FIG. 2B, an exemplary target glucose exposure determination process 208 is shown, according to one embodiment of the present disclosure. In multiple embodiments, in order to determine a target glucose exposure for a particular hour, the system 100 may first divide a glucose exposure limit by 24 to get a glucose exposure limit per hour. In one or more embodiments, the glucose exposure limit may be the maximum amount of glucose exposure the user desires over the course of a twenty-four hour day. In at least one embodiment, the user may input the glucose exposure limit into the system 100. For example, in one embodiment, the user may input a glucose exposure limit of 1200 units of measurement of glucose exposure into the system 100, which the system 100 divides by 24 to determine that the glucose exposure limit per hour is 50 units of measurement of glucose exposure.

In various embodiments, as shown in step 214, the system 100 may multiply the glucose exposure limit per hour by a numerical expression of the particular hour. In many embodiments, the numerical expression of the particular hour correlates to the particular time of day, using a 00:00-24:00 time measure for the time of day (or 00:01-24:00 or another suitable similar range). For example, in one embodiment, the particular hour 11:00 AM correlates to 11 for the numerical expression of the particular hour. In a further embodiment, the minutes portion of the time of day correlates to a decimal for the numerical expression of the particular hour. For example, in the further embodiment, the time of day 5:15 PM correlates to 17.25 for the numerical expression of the particular hour.

According to one embodiment, as an example of steps 212 and 214, in multiple embodiments, the user may input a glucose exposure limit of 1800 units of measurement. In many embodiments, the system 100 may then divide the glucose exposure limit by 24, to get a glucose exposure limit per hour of 75 units of measurement per hour. Next, in several embodiments, if the particular hour is 3:00 PM, the system 100 may multiple the glucose exposure limit per hour by the numerical expression of 3:00 PM, which is 15. In one or more embodiments, the system 100 may determine that the target glucose exposure for 3:00 PM is 75 units of measurement per hour multiplied by 15 hours, which is 1125 units of measurement of glucose exposure.

In an alternative embodiment, the system 100 may divide the glucose exposure limit by 1440 to get a glucose exposure limit per minute. Continuing with this alternative embodiment, the system 100 may multiply the glucose exposure limit per minute by a numerical expression of a particular minute. In this alternative embodiment, the particular minute may be a specific minute during the day such that the numerical expression of the particular minute is between 0 and 1440. For example, still continuing in the alternative embodiment, at 1:45 PM, the particular minute is equal to thirteen hours multiplied by 60, and then added to the remaining 45 minutes, which is 825 minutes. In various embodiments, the system 100 may display the glucose exposure as a proportion to the target glucose exposure for the particular minute. In a further embodiment, similar calculations may be done so that the system 100 may determine a target glucose exposure for a particular second.

In a further embodiment, the glucose exposure limit may be a function of the user's personal information, such as, but not limited to, the user's height, weight, body mass index score, average daily exercise, average daily glucose exposure, whether the user is preparing for an endurance race, and/or other similar information. In this embodiment, the system 100 may calculate a healthy glucose exposure limit, based on algorithms and based on the user's personal health goals. For example, in one embodiment, the user may want to lose weight, so the user may input a “lose weight” goal into the system 100, and based on the user's personal information and other factors, the system 100 determines a glucose exposure limit for the user.

In a further embodiment, the system 100 may import or receive data from other devices that determine data about a user (or about other users). In various embodiments, the system 100 may store system 100 data in a server with other system 100 data for other users. In one or more embodiments, system 100 data may include the user's personal information, as well as the user's historical glucose data. In at least one embodiment, if a user updates the user's personal information and the update includes a change in body mass index score or weight, the system 100 may determine if the user has increased or decreased glucose exposure. In a further embodiment, the system 100 may deploy machine learning or AI to optimize the glucose exposure limits for a variety of user body types, by using measured glucose data against increases and decreases in users' weight and body mass index scores.

In various embodiments, the system 100, at step 208, may receive, from the user or the system, a glucose exposure limit per hour. In several embodiments, the glucose exposure limit per hour may be utilized to calculate the target glucose exposure for a particular hour by multiplying the glucose exposure limit per hour by the numerical expression of the particular hour, as discussed infra. In one or more embodiments, the glucose exposure limit per hour may be multiplied by 24 to get a 24-hour glucose exposure limit. For example, in one embodiment, the user or the system may provide a glucose exposure limit per hour of 80 mg/dL, which the system may then multiply by 24 to determine the glucose exposure limit. Continuing with the example, in some embodiments, if the user checks his glucose exposure at 3:00 PM, and the 24-hour period began at midnight (00:00 AM), the system would multiply the glucose exposure limit per hour by 15 to get the target glucose exposure for the particular hour (1200 mg/dL). Still continuing with the above example, in many embodiments, the system may thereafter compare the user's glucose exposure with the target glucose exposure for the particular hour, and may also display the 24-hour glucose exposure limit.

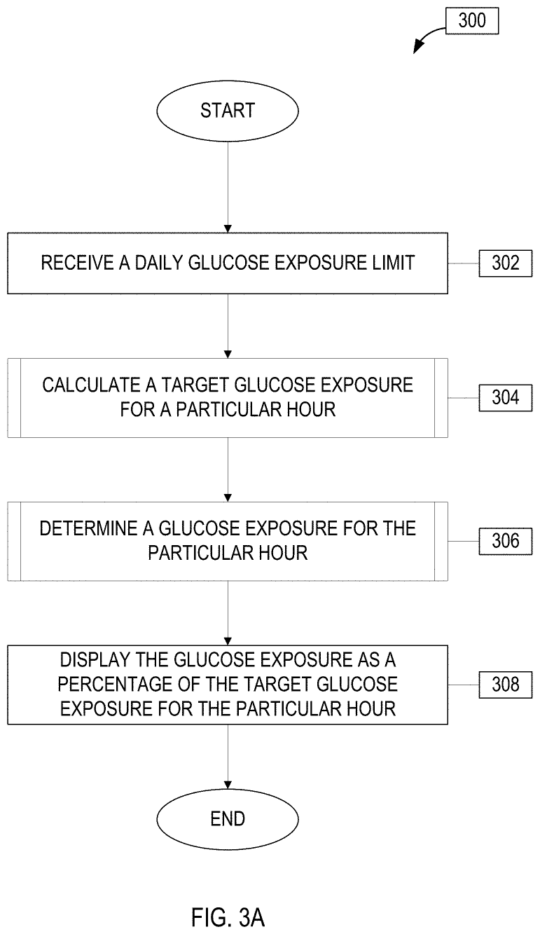

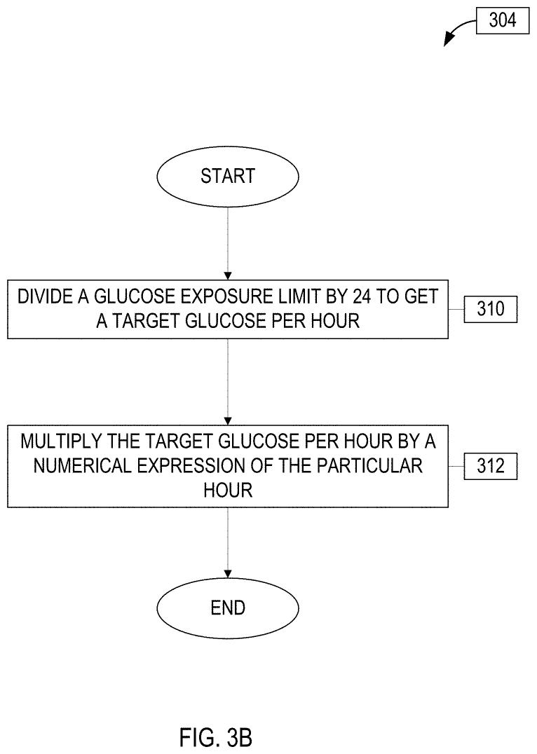

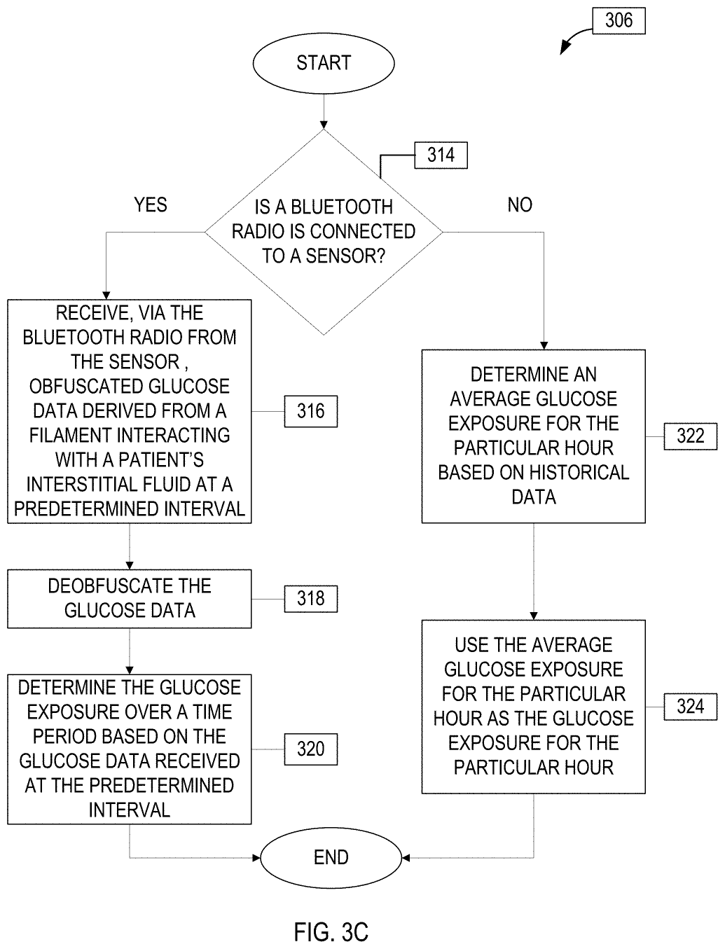

An exemplary glucose exposure process 300 is shown in FIG. 3A, according to one embodiment of the present disclosure. In various embodiments, a user may first connect the sensor 104 to the user's body, such that the glucose sensor 120 is interacting with the interstitial fluid or other bodily fluid.

As shown in step 302 of process 300, in multiple embodiments, the system 100 may receive a daily glucose exposure limit. In one or more embodiments, the user may input the daily glucose exposure limit into the system 100 via the device 106 or the computing device 124. In at least one embodiment, the daily glucose exposure limit may be the maximum amount of glucose exposure the user desires to receive over the course of a twenty-four hour day. In one or more embodiments, a medical professional or other third-party may input the daily glucose exposure limit into the system 100. In a further embodiment, a physician or other medical professional may prescribe a specific daily glucose exposure limit for the user. In some embodiments, the system 100 may calculate the glucose exposure limit based on weight loss goals, machine learning and artificial intelligence, physical activity goals, or other calculations.

At step 304, in various embodiments, the system 100 may calculate a target glucose exposure for a particular hour. As discussed in more detail below (in reference to FIG. 3B), in some embodiments, step 304 includes dividing a glucose exposure limit by 24 to get a glucose exposure limit per hour (step 310) and multiplying the glucose exposure limit per hour by a numerical expression of the particular hour (step 312). In one or more embodiments, the target glucose exposure for the particular hour may be the amount of glucose exposure the user is trying to attain for the particular hour. In many embodiments, the target glucose exposure for the particular hour may be a limit of glucose exposure that the user is trying not to exceed. In at least one embodiment, the particular hour may be a specific time during a twenty-four hour period. For example, in one embodiment, the particular hour may be 9:00 AM.

As shown in step 306, in several embodiments, the system 100 may determine a glucose exposure for the particular hour. As discussed in more detail below (in reference to FIG. 3C), in some embodiments, step 306 includes determining if a Bluetooth radio is connected to the sensor 104 (step 314), and if so, receiving, via the Bluetooth radio from the sensor 104, obfuscated glucose data (step 316), deobfuscating the data (step 318), and determining the glucose exposure over a time period based on the glucose data received at the predetermined interval (step 320), and if the Bluetooth radio is not connected to the sensor, determining an average glucose exposure for the particular hour based on historical data (step 322), and using the average glucose exposure for the particular hour as the glucose exposure for the particular hour (step 324).

At step 308, in many embodiments, the system 100 may display the glucose exposure calculated at step 306 as a percentage of the target glucose exposure for the particular hour calculated at step 304. For example, in one embodiment, the glucose exposure calculated at step 306 may be 1140 at the particular hour, and the target glucose exposure for the particular hour is 1080, which would be displayed as 105.5%.

As described in FIG. 3B, an exemplary target glucose exposure determination process 304 is shown, according to one embodiment of the present disclosure. As described in step 310, in multiple embodiments, in order to determine a target glucose exposure for a particular hour, the system 100 may first divide the daily glucose exposure limit by 24 to get a glucose exposure limit per hour. For example, in one embodiment, the user may input a glucose exposure limit of 1200 units of measurement of glucose exposure into the system 100, which the system 100 divides by 24 to determine that the glucose exposure limit per hour is 50 units of measurement of glucose exposure.

In various embodiments, as shown in step 312, the system 100 may multiply the glucose exposure limit per hour by a numerical expression of the particular hour. In many embodiments, the numerical expression of the particular hour correlates to the particular time of day, using a 00:00-24:00 time measure for the time of day. For example, in one embodiment, the particular hour 11:00 AM correlates to 11 for the numerical expression of the particular hour. In a further embodiment, the minutes portion of the time of day correlates to a decimal for the numerical expression of the particular hour. For example, in the further embodiment, the time of day 5:15 PM correlates to 17.25 for the numerical expression of the particular hour.

According to one embodiment, as an example of steps 310 and 312, in multiple embodiments, the user may input a glucose exposure limit of 1800 units of measurement. In many embodiments, the system 100 may then divide the glucose exposure limit by 24, to get a glucose exposure limit per hour of 75 units of measurement per hour. Next, in several embodiments, if the particular hour is 3:00 PM, the system 100 may multiple the glucose exposure limit per hour by the numerical expression of 3:00 PM, which is 15. In one or more embodiments, the system 100 may determine that the target glucose exposure for 3:00 PM is 75 units of measurement per hour multiplied by 15 hours, which is 1125 units of measurement of glucose exposure.

In various embodiments, the system 100, at step 304, may receive, from the user or the system, a glucose exposure limit per hour. In several embodiments, the glucose exposure limit per hour may be utilized to calculate the target glucose exposure for a particular hour by multiplying the glucose exposure limit per hour by the numerical expression of the particular hour, as discussed infra. In one or more embodiments, the glucose exposure limit per hour may be multiplied by 24 to get a 24-hour glucose exposure limit. For example, in one embodiment, the user or the system may provide a glucose exposure limit per hour of 80 mg/dL, which the system may then multiply by 24 to determine the glucose exposure limit. Continuing with the example, in some embodiments, if the user checks his glucose exposure at 3:00 PM, and the 24-hour period began at midnight (00:00 AM), the system would multiply the glucose exposure limit per hour by 15 to get the target glucose exposure for the particular hour (1200 mg/dL). Still continuing with the above example, in many embodiments, the system may thereafter compare the user's glucose exposure with the target glucose exposure for the particular hour, and may also display the 24-hour glucose exposure limit.

FIG. 3C shows an exemplary glucose exposure for a particular hour determination process 306, according to one embodiment of the present disclosure. In various embodiments, at step 314 of process 306, the system 100 determines whether a Bluetooth radio is connected to the sensor 104. In one or more embodiments, the system 100 is wirelessly connected to the sensor 104.

In multiple embodiments, as shown in step 316, if a Bluetooth radio is connected to the sensor 104, then the system 100 receives, via the Bluetooth radio from the sensor 104, obfuscated glucose data derived from the glucose sensor 120 interacting with a patient's interstitial fluid at a predetermined interval. In one or more embodiments, the predetermined interval may be the time between the system 100 receiving glucose data from the sensor 104. In at least one embodiment, the predetermined interval may be one second, such that the system 100 is essentially constantly receiving obfuscated glucose data. In some embodiments, the predetermined interval may be one hour, such that the system 100 receives obfuscated glucose data once per hour. In many embodiments, the predetermined interval may be one day or multiple days.

In an embodiment, the sensor 104 may transmit a batch of readings from the glucose sensor 120 to the system 100 instead of transmitting each individual reading upon receiving the reading from the glucose sensor 120. For example, in this embodiment, the glucose sensor 120 may send the sensor 104 the glucose data, as described above, at a constant rate (e.g., one reading per second), but the sensor 104 may collect a certain amount of glucose data from multiple readings (a batch) from the glucose sensor 120 (e.g., every five, ten, twenty readings) before transmitting the batch of glucose data to the system 100. In some embodiments, the batch may include an amount of readings from the glucose sensor 120 to the sensor 104 over a period of time (e.g., amount of readings per hour). Still continuing with this embodiment, the predetermined interval may be the amount of time between the glucose sensor 120 sending readings to the sensor 104 or may be the amount of time between the sensor 104 sending batches of readings to the system 100.

As described in step 318, in various embodiments, the system 100 deobfuscates the glucose data. In one or more embodiments, the device 106, the data collection server 108, or the computing device 124 may deobfuscate the glucose data. In some embodiments, the device 106 may receive the obfuscated glucose data via Bluetooth radio or other similar communication device. In at least one embodiment, the device 106, after receiving the obfuscated glucose data, may deobfuscate the glucose data or send the obfuscated glucose data to the data collection server 108, which will deobfuscate the glucose data. In one embodiment, if the device 106 sends the obfuscated glucose data to the data collection server 108, the data collection server 108 (or the connected computing device 124) may deobfuscate the glucose data, and the data collection server 108 may thereafter transmit the deobfuscated glucose data to the device 106. In many embodiments, once the glucose data is deobfuscated, the system 100 may read and utilize the glucose data/information.

As shown in step 320, in several embodiments, the system 100 determines the glucose exposure over a time period based on the glucose data received at the predetermined interval. In at least one embodiment, the glucose exposure over a time period may be an average of the glucose data received at the predetermined interval over the course of the time period.

In many embodiments, the time period (or time interval) may be an overall amount of time from which the glucose exposure is being measured. For example, in one embodiment, the system 100 may determine the glucose exposure at the time 8:00 AM (the particular hour). Continuing with the example, in some embodiments, the time period may be from 00:00 AM to 8:00 AM, such that the system 100 determines the glucose exposure for the time period.

In another example, in at least one embodiment, the system 100 may determine the glucose exposure at the time 8:00 AM (the particular hour), and the time period may be one hour. Continuing with this example, the system 100 may determine the glucose exposure for each time period, and determine the glucose exposure at 8:00 AM by summing up the individual glucose exposures for each hour (or other increment of time) throughout the day. In one or more embodiments, the time period may range from one second to one day (such as, e.g., 15 minutes, 30 minutes, 1 hour, 3 hours, 1 day, etc.).

For example, in one embodiment, the predetermined interval may be one minute, and the time period may be thirty minutes, such that the system 100 receives glucose data thirty times within the time period. Continuing with the example, in at least one embodiment, the system 100 may calculate the average of the thirty glucose data points to determine the glucose exposure over the period of time. In an alternate embodiment, the system 100 may calculate a weighted average of the thirty glucose data points, such that the later received glucose data points have more weight than the earlier received glucose data points.

In a further embodiment, the system 100 may calculate a running total of glucose exposure through a twenty-four hour day by adding the determined glucose exposures over the time periods throughout the twenty-four hours. For example, in one embodiment, if the time period is sixty minutes, and the twenty-four hour day begins at midnight (00:00 AM), the system 100 may add each glucose exposure (in units of measurement) for each sixty minute time period over the course of the twenty-four hour day, so that, at a particular hour (e.g., 9:00 AM), the system 100 may determine the total glucose exposure for the user for the day at 9:00 AM.

In one or more embodiments, the system may be configured to compensate for a disconnected sensor and may use one or more smoothing algorithms (or the like) to fill in or approximate glucose exposure for an hour (or other suitable time period). For example, if a user is sleeping and is not wearing a sensor, the system may use historical or other data/stored information to estimate the user's glucose exposure while the sensor is disconnected.

At step 322, in multiple embodiments, if a Bluetooth radio/receiver is not connected to the sensor 104, the system 100 determines an average glucose exposure for the particular hour based on historical data. In this embodiment, since the system 100 is not connected to the sensor 104, the system 100 may not be able to receive current glucose data at the predetermined interval from the sensor 104. In many embodiments, the system 100 may store historical glucose data such that the system 100 may retrieve historical glucose data from previous days and utilize the historical data in the present average glucose exposure determination. In one or more embodiments, the utilization of the historical data allows the system 100 to continue to calculate the total glucose exposure and display the glucose exposure as a percentage of the target glucose exposure for the particular hour. In at least one embodiment, the historical data may include particular hour information, such that the system 100 may incorporate historical data from the same particular hour as the particular hour glucose data that is missing due to the system 100 not being connected to the sensor 104.

For example, in one embodiment, the Bluetooth radio may not be connected to the sensor 104 from 2:00 PM to 3:00 PM. Continuing with the example, in several embodiments, the system 100 may retrieve stored historical data from 2:00 PM to 3:00 PM from previous days, and average the stored historical data for the particular hour to get an average glucose exposure for the particular hour based on historical data (an average glucose level based on historical/stored data). In an alternative embodiment, the system 100 may determine a weighted average for the glucose exposure for the particular hour based on historical data, such that the more recent historical data is given more weight than the older historical data, because the more recent historical data is more likely to be more accurate to the actual current glucose exposure.

In at least one embodiment, if the system 100 does not receive the glucose data from the sensor 104 at the particular interval, the system 100 may apply one or more smoothing algorithms once the system 100 is reconnected to the sensor 104, to back fill the missing glucose data. In one or more embodiments, the one or more smoothing algorithms may include calculating an average glucose exposure based on the glucose data received before and after the system 100 stopped receiving glucose data from the sensor 104. For example, in one embodiment, if the system 100 did not receive glucose data for one predetermined interval, the system 100 may utilize immediately preceding glucose data for at least one predetermined interval and immediately succeeding glucose data for at least one predetermined interval, and average the at least two glucose data points together to determine the missing glucose data for the predetermined interval. In at least one embodiment, the system 100 may utilize multiple immediately preceding glucose data points and multiple immediately succeeding glucose data points to determine the missing glucose data for the predetermined interval. In many embodiments, the user's glucose exposure does not vary much from one predetermined interval to the next, so the system 100 is able to take an average from the glucose data from preceding and succeeding glucose data to fill in a missing glucose data point with a high level of accuracy.

In one or more embodiments, the system 100 may utilize a combination of historical data and an average of recent data to determine missing glucose data points. For example, in one embodiment, if the system 100 is missing a glucose data point for 10:00 AM, the system 100 may retrieve historical glucose exposure data for 10:00 AM for the user, as well as calculate an average of recent preceding and succeeding glucose data, and determine or estimate the missing glucose data point from a combination of the historical glucose exposure data and the average of recent preceding and succeeding glucose data. In a further embodiment, the system 100 may also utilize other users' glucose exposure data to determine missing glucose data points. In this further embodiment, the system 100 may recognize other users as similar to the user with missing glucose exposure data, based on similarities in the users' profiles, such as the users' age, gender, height, weight, similarity in glucose exposure, and other similar factors.

In various embodiments, the system 100 may notify the user if the system 100 determines that the user's glucose exposure is higher or lower than the target glucose exposure for a particular hour by a more than a certain percentage. For example, in at least one embodiment, the system 100 may notify the user if the user's glucose exposure is ten percent (or more) greater than or less than the user's target glucose exposure for a particular hour. In many embodiments, the system 100 may notify the user via displaying a message on the device 106 or causing a push notification, SMS message, email, or other similar communication to display on or transmit to a secondary device.

In one or more embodiments, if the user's glucose exposure is greater than the user's target glucose for a particular hour, the system 100 may make recommendations to the user so that the user's glucose exposure may decrease in forthcoming hour(s). In some embodiments, the recommendations may include, but are not limited to, eating low-carbohydrate foods for the user's next meal, exercising, including a specific intensity level of exercising (such as, e.g., walking, jogging, running) taking insulin (for diabetic users), including rapid-acting insulin, short-acting insulin, intermediate-acting insulin, mixed-insulin, and long-acting insulin, or a combination of recommendations. In one embodiment, if the user has indicated to the system that the user's goal is to intake carbohydrates in preparation for future physical activity (such as, e.g., running a marathon), the system 100 may not notify the user if the user's glucose exposure exceeds the glucose target for a particular hour.

In multiple embodiments, the if the user's glucose exposure is less than the user's target glucose exposure for a particular hour by a certain amount, the system 100 may recommend the user take an action to increase the user's glucose exposure. In some embodiments, situations in which the system 100 may notify the user to increase the user's glucose exposure may include, but is not limited to, the user ingesting carbohydrates in preparation for a physical activity (such as, e.g., a triathlon), hypoglycemia, or other situations in which the user's glucose exposure is lower than the target glucose exposure for a particular hour. In many embodiments, the system 100 may recommend to a user to eat a carbohydrate-rich meal or snack to increase the user's glucose exposure if the user's glucose exposure is less than the target glucose exposure for a particular hour. For example, in one embodiment, a user may indicate to the system 100 that the user is attempting to reach or surpass the target glucose exposure for a particular hour in preparation to run a marathon, and so if the user's glucose exposure is five percent lower than the target glucose exposure for a particular hour, the system 100 may notify the user and recommend the user ingest carbohydrates. In at least one embodiment, the system 100 may recommend the user seek medical treatment, such as but not limited to, going to an emergency room or calling an ambulance, or other similar medical treatment, if the user's glucose exposure is low enough to be considered hypoglycemic.

From the foregoing, it will be understood that various aspects of the processes described herein are software processes that execute on computer systems that form parts of the system. Accordingly, it will be understood that various embodiments of the system described herein are generally implemented as specially-configured computers including various computer hardware components and, in many cases, significant additional features as compared to conventional or known computers, processes, or the like, as discussed in greater detail herein. Embodiments within the scope of the present disclosure also include computer-readable media for carrying or having computer-executable instructions or data structures stored thereon. Such computer-readable media can be any available media which can be accessed by a computer, or downloadable through communication networks. By way of example, and not limitation, such computer-readable media can comprise various forms of data storage devices or media such as RAM, ROM, flash memory, EEPROM, CD-ROM, DVD, or other optical disk storage, magnetic disk storage, solid state drives (SSDs) or other data storage devices, any type of removable non-volatile memories such as secure digital (SD), flash memory, memory stick, etc., or any other medium which can be used to carry or store computer program code in the form of computer-executable instructions or data structures and which can be accessed by a computer.

When information is transferred or provided over a network or another communications connection (either hardwired, wireless, or a combination of hardwired or wireless) to a computer, the computer properly views the connection as a computer-readable medium. Thus, any such a connection is properly termed and considered a computer-readable medium. Combinations of the above should also be included within the scope of computer-readable media. Computer-executable instructions comprise, for example, instructions and data which cause a computer to perform one specific function or a group of functions.

Those skilled in the art will understand the features and aspects of a suitable computing environment in which aspects of the disclosure may be implemented. Although not required, some of the embodiments of the claimed systems and processes may be described in the context of computer-executable instructions, such as program modules or engines, as described earlier, being executed by computers in networked environments. Such program modules are often reflected and illustrated by flow charts, sequence diagrams, exemplary screen displays, and other techniques used by those skilled in the art to communicate how to make and use such computer program modules. Generally, program modules include routines, programs, functions, objects, components, data structures, application programming interface (API) calls to other computers whether local or remote, etc. that perform particular tasks or implement particular defined data types, within the computer. Computer-executable instructions, associated data structures and/or schemas, and program modules represent examples of the program code for executing steps of the methods disclosed herein. The particular sequence of such executable instructions or associated data structures represent examples of corresponding acts for implementing the functions described in such steps.

Those skilled in the art will also appreciate that the claimed and/or described systems and methods may be practiced in network computing environments with many types of computer system configurations, including personal computers, smartphones, tablets, hand-held devices, multi-processor systems, microprocessor-based or programmable consumer electronics, networked PCs, minicomputers, mainframe computers, and the like. Embodiments of the claimed systems and processes are practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing various aspects of the described operations, which is not illustrated, includes a computing device including a processing unit, a system memory, and a system bus that couples various system components including the system memory to the processing unit. The computer will typically include one or more data storage devices for reading data from and writing data to. The data storage devices provide nonvolatile storage of computer-executable instructions, data structures, program modules, and other data for the computer.

Computer program code that implements the functionality described herein typically comprises one or more program modules that may be stored on a data storage device. This program code, as is known to those skilled in the art, usually includes an operating system, one or more application programs, other program modules, and program data. A user may enter commands and information into the computer through keyboard, touch screen, pointing device, a script containing computer program code written in a scripting language or other input devices (not shown), such as a microphone, etc. These and other input devices are often connected to the processing unit through known electrical, optical, or wireless connections.

The computer that effects many aspects of the described processes will typically operate in a networked environment using logical connections to one or more remote computers or data sources, which are described further below. Remote computers may be another personal computer, a server, a router, a network PC, a peer device or other common network node, and typically include many or all of the elements described above relative to the main computer system in which the systems and processes are embodied. The logical connections between computers include a local area network (LAN), a wide area network (WAN), virtual networks (WAN or LAN), and wireless LANs (WLAN) that are presented here by way of example and not limitation. Such networking environments are commonplace in office-wide or enterprise-wide computer networks, intranets, and the Internet.

When used in a LAN or WLAN networking environment, a computer system implementing aspects of the systems and processes is connected to the local network through a network interface or adapter. When used in a WAN or WLAN networking environment, the computer may include a modem, a wireless link, or other mechanisms for establishing communications over the wide area network, such as the Internet. In a networked environment, program modules depicted relative to the computer, or portions thereof, may be stored in a remote data storage device. It will be appreciated that the network connections described or shown are exemplary and other mechanisms of establishing communications over wide area networks or the Internet may be used.

While various aspects have been described in the context of a preferred embodiment, additional aspects, features, and methodologies of the claimed systems and processes will be readily discernible from the description herein, by those of ordinary skill in the art. Many embodiments and adaptations of the disclosure and claimed systems and processes other than those herein described, as well as many variations, modifications, and equivalent arrangements and methodologies, will be apparent from or reasonably suggested by the disclosure and the foregoing description thereof, without departing from the substance or scope of the claims. Furthermore, any sequence(s) and/or temporal order of steps of various processes described and claimed herein are those considered to be the best mode contemplated for carrying out the claimed systems and processes. It should also be understood that, although steps of various processes may be shown and described as being in a preferred sequence or temporal order, the steps of any such processes are not limited to being carried out in any particular sequence or order, absent a specific indication of such to achieve a particular intended result. In most cases, the steps of such processes may be carried out in a variety of different sequences and orders, while still falling within the scope of the claimed systems and processes. In addition, some steps may be carried out simultaneously, contemporaneously, or in synchronization with other steps.

The embodiments were chosen and described in order to explain the principles of the claimed systems and processes and their practical application so as to enable others skilled in the art to utilize the systems and processes and various embodiments and with various modifications as are suited to the particular use contemplated. Alternative embodiments will become apparent to those skilled in the art to which the claimed systems and processes pertain without departing from their spirit and scope. Accordingly, the scope of the claimed systems and processes is defined by the appended claims rather than the foregoing description and the exemplary embodiments described therein.

1. A glucose exposure process comprising:
receiving a glucose exposure limit per hour;
determining a target glucose for a particular hour by multiplying the glucose exposure limit per hour by a numerical representation of the particular hour;
determining a glucose exposure for the particular hour by performing operations comprising:
upon determining that a receiver is not connected to a sensor:
determining an average glucose exposure for the particular hour based on stored information; and
determining the glucose exposure for the particular hour based on the average glucose exposure for the particular hour;
determining a total of the glucose exposure by adding the glucose exposure for the particular hour to a summation of glucose exposure for time intervals preceding the particular hour; and
displaying the total of the glucose exposure as a proportion of the target glucose for the particular hour.

2. The glucose exposure process of claim 1, wherein determining the average glucose exposure for the particular hour based on the stored information comprises averaging a glucose exposure for an hour immediately preceding the particular hour and an hour immediately following the particular hour.

3. The glucose exposure process of claim 1, wherein determining the average glucose exposure for the particular hour based on the stored information comprises:
retrieving stored glucose exposure information for the particular hour; and
averaging the stored glucose exposure information for the particular hour.

4. The glucose exposure process of claim 3, wherein the stored glucose exposure information is stored in memory at a cloud-based device.

5-19. (canceled)

20. A system comprising:
a processor; and
a non-transitory computer-readable medium having instructions stored thereon, the instructions executable by the processor for performing operations comprising:
receiving a glucose exposure limit per hour;
determining a target glucose for a particular hour by multiplying the glucose exposure limit per hour by a numerical representation of the particular hour;
determining a glucose exposure for the particular hour by performing further operations comprising:
upon determining that a receiver is not connected to a sensor:
determining an average glucose exposure for the particular hour based on stored information; and
determining the glucose exposure for the particular hour based on the average glucose exposure for the particular hour;
determining a total of the glucose exposure by adding the glucose exposure for the particular hour to a summation of glucose exposure for time intervals preceding the particular hour; and
displaying the total of the glucose exposure as a proportion of the target glucose for the particular hour.

21. The system of claim 20, wherein the operation of determining the average glucose exposure for the particular hour based on the stored information comprises averaging a glucose exposure for an hour immediately preceding the particular hour and an hour immediately following the particular hour.

22. The system of claim 20, wherein the operation of determining the average glucose exposure for the particular hour based on the stored information comprises:
retrieving stored glucose exposure information for the particular hour; and
averaging the stored glucose exposure information for the particular hour.

23. The system of claim 22, wherein the stored glucose exposure information is stored in memory at a cloud-based device.

24. A non-transitory computer-readable medium having program code that is stored thereon, the program code executable by one or more processing devices for performing operations comprising:
receiving a glucose exposure limit per hour;
determining a target glucose for a particular hour by multiplying the glucose exposure limit per hour by a numerical representation of the particular hour;
determining a glucose exposure for the particular hour by performing further operations comprising:
upon determining that a receiver is not connected to a sensor:
determining an average glucose exposure for the particular hour based on stored information; and
determining the glucose exposure for the particular hour based on the average glucose exposure for the particular hour;
determining a total of the glucose exposure by adding the glucose exposure for the particular hour to a summation of glucose exposure for time intervals preceding the particular hour; and
displaying the total of the glucose exposure as a proportion of the target glucose for the particular hour.

25. The non-transitory computer-readable medium of claim 24, wherein the operation of determining the average glucose exposure for the particular hour based on the stored information comprises averaging a glucose exposure for an hour immediately preceding the particular hour and an hour immediately following the particular hour.

26. The non-transitory computer-readable medium of claim 24, wherein the operation of determining the average glucose exposure for the particular hour based on the stored information comprises:
retrieving stored glucose exposure information for the particular hour; and
averaging the stored glucose exposure information for the particular hour.

27. The non-transitory computer-readable medium of claim 26, wherein the stored glucose exposure information is stored in memory at a cloud-based device.
